(19) United States
(12) Patent Application Publication
(10) Pub. No.: US 2025/0290891 A1
(43) Pub. Date: Sep. 18, 2025

(54) SYSTEMS AND METHODS FOR POINT-OF-CARE ELECTROCHEMICAL DETECTION OF BIOMARKERS IN LIQUIDS

(71) Applicants: Alfred ANDAMA, Kampala (UG); Devan JAGANATH, San Francisco, CA (US); Adithya CATTAMANCHI, San Francisco, CA (US); UNIVERSITY OF UTAH RESEARCH FOUNDATION, Salt Lake City, UT (US)

(72) Inventors: Swomitra Kumar MOHANTY, Salt Lake City, UT (US); Christina Nicole CHRISTENSEN, Salt Lake City, UT (US); Shaylee LARSON, Salt Lake City, UT (US); Mary JEPPSON, Salt Lake City, UT (US); Zachary RASMUSSEN, Salt Lake City, UT (US); Alfred ANDAMA, Kampala (UG); Devan JAGANATH, San Francisco, CA (US); Adithya CATTAMANCHI, San Francisco, CA (US)

(73) Assignee: UNIVERSITY OF UTAH RESEARCH FOUNDATION, Salt Lake City, UT (US)

(21) Appl. No.: 19/082,655

(22) Filed: Mar. 18, 2025

Related U.S. Application Data
(60) Provisional application No. 63/566,538, filed on Mar. 18, 2024.

(51) Int. Cl. G01N 27/327; A61B 5/097; G01N 33/487; G06N 20/00; G16H 10/40; G16H 50/20

(52) U.S. Cl. CPC G01N 27/3277; A61B 5/097; G01N 33/48707; G06N 20/00; G16H 10/40; G16H 50/20

(57) ABSTRACT

Electrochemical detection systems and methods employ square wave voltammetry (SWV) to analyze biomarker interactions with an engineered electroactive solution containing transition metal ions, e.g., Cu²⁺ or Co²⁺. The system is designed to detect clinically relevant volatile organic biomarkers and compounds (VOBs and VOCs) linked to infections, e.g., methyl nicotinate for tuberculosis, and other respiratory or metabolic conditions. Exhaled breath from a patient is passively collected on a substrate of a mask over a predefined wear period. Any VOCs are collected as part of this breath sample as well. A defined region including the patient sample is then excised and transferred to an electrochemical cell containing an engineered electroactive solution for SWV analysis. The VOCs undergo redox reactions, generating electrochemical signatures that serve as fingerprints for disease-specific biomarkers and enable in-the-field diagnoses. The methodology is adaptable for point-of-care diagnostics, offering a rapid, portable, and low-cost alternative to conventional gas chromatography-mass spectrometry.

### CROSS-REFERENCE TO RELATED APPLICATIONS

This application claims the benefit of U.S. Provisional Patent Application No. 63/566,538, filed Mar. 18, 2024, which is incorporated by reference as if disclosed herein in its entirety.

### STATEMENT REGARDING FEDERALLY SPONSORED RESEARCH AND DEVELOPMENT

This invention was made with government support under R01 HL139717 awarded by the National Institutes of Health. The government has certain rights in the invention.

### BACKGROUND

Tuberculosis (TB) remains a significant global health issue. The World Health Organization's (WHO) 2021 Global Tuberculosis Report revealed that approximately 1.5 million people died of TB in 2020, with the majority of cases occurring in Southeast Asia and Africa. Although often treatable with a standard antibiotic regimen, many people lack access to diagnosis and treatment, resulting in an estimated one-third of TB cases going unreported. In high-income countries, the time from symptom onset to treatment initiation averages 25 days, while in low-income countries, the time to treatment averages 56 days. Numerous healthcare provider visits and diagnostic tests are often performed before patients receive a diagnosis. Additionally, drug-resistant TB has emerged as a growing health concern since 2020, increasing morbidity, mortality, and the urgency for prompt treatment to prevent disease transmission.

To address this public health emergency, the WHO launched the “End TB” initiative, aiming to reduce the global average time from TB symptom onset to treatment to less than one month by 2025. A factor in achieving this goal is improving diagnostic access.

However, current diagnostic methods are expensive, time-consuming, and use sophisticated equipment and training to administer. For example, existing diagnostic solutions are typically culture, polymerase chain reaction (PCR), and/or antibody-based approaches, most of which utilize a central medical facility for operation. Primary TB diagnostic methods include sputum smear microscopy, Xpert MTB/RIF test, chest X-rays, and immunological tests (TST and IGRAs). Although sputum smear microscopy was the most common diagnostic method, it has low sensitivity, particularly in children, ranging from 20-80% based on extrapulmonary infection spread. The Xpert MTB/RIF test, recently recommended by WHO, offers higher sensitivity, rifampicin resistance testing, and rapid results, but also utilizes costly equipment and a stable power supply. Chest X-rays, typically employed when initial methods are inconclusive, utilize special equipment and radiologist interpretation. TST and IGRAs detect immune responses to TB, but their results take days to obtain and can be confounded by the BCG TB vaccine.

Researchers are now focusing on developing affordable point-of-care devices, such as non-invasive breath biomarker detectable sensors, aiming to meet the WHO target product profile with ≥90% sensitivity and ≥70% specificity. This approach could provide an early, rapid, and cost-effective diagnosis to millions who otherwise would not receive treatment. As a result, detecting volatile organic biomarkers (VOBs) in breath has led to increasing interest in medical diagnostics.

Existing solutions for many of these diseases utilize complex, infrastructure, are not suitable for point-of-care, and/or are invasive.

While current commercial products show promise, they have yet to meet the WHO's sensitivity and specificity requirements and are not targeted for the detection of specific VOBs. Traditionally, techniques such as gas chromatography-mass spectrometry (GC-MS) have been used to identify and correlate VOBs (or Volatile Organic Compounds (VOCs)) to disease states.

What is desired therefore is a device to enable early, accurate, and inexpensive point-of-care detection of specific VOBs and VOCs in a patient sample for effective treatment, e.g., of TB.

### SUMMARY

Aspects of the present disclosure are directed to non-invasive diagnostic systems and methods for the capture and electrochemical analysis of volatile organic biomarkers (VOBs) and Volatile Organic Compounds (VOCs). Some embodiments of the present disclosure utilize a mask-based collection system to collect breath exhaled by individuals. In some embodiments, the system includes a standard face mask that passively collects VOCs from exhaled breath over a wear time of, e.g., 20 to 60 minutes. After use, a two-inch circular section can be cut from the mask in the region corresponding to the wearer's mouth and transferred into an electroactive solution to facilitate VOC desorption. The solution can then be analyzed using square wave voltammetry (SWV) to detect and quantify specific biomarkers indicative of disease states, such as tuberculosis or respiratory infections. This process results in a digital data set with peaks that are directly related to oxidation and reduction reactions that occur in the testing solution. In some embodiments, the data set is used in a machine learning algorithm, e.g., that is designed to predict patients' status for a disease. Embodiments of the present disclosure leverage engineered electroactive solutions with transition metal ions, such as copper (II) salts, to enable the selective detection of target VOCs. The methodology is adaptable for point-of-care diagnostics, offering a rapid, portable, and low-cost alternative to conventional gas chromatography-mass spectrometry (GC-MS).

Aspects of the present disclosure are directed to a system for point-of-care screening of a patient for a condition. In some embodiments, the system includes a solution reservoir including a volume of an electroactive solution. In some embodiments, the system includes a sample collector configured to collect a patient sample directly from a human patient and transport the patient sample to the electroactive solution. In some embodiments, the patient sample includes breath, urine, saliva, sweat, odor, or combinations thereof. In some embodiments, the sample collector includes a mask, an absorbent substrate, a conduit, or combinations thereof. In some embodiments, the sample collector includes a mask with an absorbent substrate reversibly attached thereto, wherein the mask is configured to direct patient sample collected directly from the patient to the absorbent substrate. In some embodiments, the system includes a pair of electrodes positioned to contact a sample mixture, the sample mixture including electroactive solution and patient sample. In some embodiments, the system includes a potentiostat in electrical contact with the pair of electrodes. In some embodiments, the system is configured to perform electrochemical analysis on the sample mixture via the pair of electrodes to produce an electrochemical data output including a distinct electrochemical signature indicative of one or more biomarkers in the sample mixture. In some embodiments, the electrochemical analysis includes amperometry, potentiometry, conductometry, SWV, or combinations thereof.

In some embodiments, the electroactive solution including a concentration of metal ions. In some embodiments, the metal ions include Li, Cu, Fe, Ni, Cr, Co, Ca, Mg, Zn, In, Al, or combinations thereof. In some embodiments, the electroactive solution includes dissolved lithium (I) metal salts, copper (I) metal salts, iron (II) metal salts, nickel (II) salts, chromium (II) metal salts, cobalt (II) metal salts, calcium (II) metal salts, magnesium (II) metal salts, zinc (II) metal salts, copper (II) metal salts, indium (III) metal salts, cobalt (III) metal salts, iron (III) metal salts, aluminum (III) metal salts, chromium (III) metal salts, or combinations thereof. In some embodiments, the metal ions are provided by copper (II) chloride. In some embodiments, the electroactive solution further comprises acetone, ethanol, I⁺, citric acid, acetic acid, or combinations thereof.

In some embodiments, the one or more biomarkers are infectious disease biomarkers, cancer biomarkers, non-communicable disease biomarkers, or combinations thereof. In some embodiments, the one or more biomarkers are tuberculosis (TB) biomarkers, pneumonia biomarkers, malaria biomarkers, schistosomiasis biomarkers, colorectal cancer biomarkers, or combinations thereof. In some embodiments, the one or more biomarkers includes methyl-nicotinate (MN); methyl p-anisate (MPA); 2-methyl butane; acrolein; furan; tetrahydro; heptane; ethylbenzene; carane; tetradecane; tetradecanal; octane; bexanal, 2,4-dimethylheptane; ethylcyclohexane: 2,4-dimethylhept-1-ene; 4-hydroxy-4-methylpentan-2-one; ethylbenzene; m-xylene, p-xylene; o-xylene; propylcyclohexane; 1-ethyl-3-methylbenzene; benzaldehyde; 1,2,4-trimethylbenzene; decane; octanal; s(−)-limonene; 2-ethylhexan-1-ol; nonanal; dodecane; phenylacetylglutamine (PAG); hippurate; 1,1-(1-butenylidene)-bis-benzene; 1,3-dimethylbenzene; 1,4-dimethylbenzene; 2-amino-5-isopropyl-8-methyl-1-azulenecarbonitrile; 1-iodononane; [(1,1-dimethylethyl)thio]acetic acid; 4-(4-propylcyclohexyl)-40-cyano[1,10-bibiphenyl]-4-yl ester benzoic acid; decanal; 4-methyl-2-pentanone; 2-pentanone; 4-methyloctane; 4-methylundecane; 2-methylpentane; 3-methylpentane; methylcyclopentane; cyclohexane; methylcyclohexane; trimethyldecane-1,2-pentadiene; cyclohexanone; 2,2-dimethyldecane; 4-ethyl-1-octyn-3-ol; trans-2-dodecen-1-ol; cyclooctylmethanol; ethylaniline; heptanal; 2-methyl-3-phenyl-2-propenal; p-cymene; 1,2-dihydro-1,1,6-trimethyl-naphthalene; 1,4,5-trimethylnaphthalene; anisole; 1-octanol; 4-methylphenol; c-terpinene; bomylene; dimethyl disulfide; 2-methoxythiophene; 2,7-dimethylquinoline; acetaldehyde; acetone; 4-heptanone; allylisothiocyanate; methoxy-phenyl-oxime; hexen-1-ol, or combinations thereof.

In some embodiments, the system includes a computer readable storage device having stored thereon instructions that when executed by one or more processors result in the following operation: training a machine learning algorithm on an electrochemical training data set to identify an electrochemical signature indicative of a particular biomarker, the electrochemical training data including a plurality of electrochemical data outputs from condition-positive patient samples and electrochemical data outputs from condition-negative patient samples. In some embodiments, the electrochemical training data set includes data obtained from a process including amperometry, potentiometry, conductometry, SWV, or combinations thereof.

Aspects of the present disclosure are directed to a device for point-of-care screening of a patient. In some embodiments, the device includes a solution reservoir including a volume of an electroactive solution; a pair of electrodes in contact with the electroactive solution; a sample collector configured to collect a patient sample directly from a human patient and transport the patient sample to the electroactive solution, the sample collector including a mask with an absorbent substrate reversibly attached thereto, wherein the mask is configured to direct patient sample collected directly from the patient to the absorbent substrate; and a potentiostat in electrical contact with the pair of electrodes. In some embodiments, the device is configured to perform electrochemical analysis on the electroactive solution after the patient sample has been mixed therein via the pair of electrodes to produce an electrochemical data output including a distinct electrochemical signature indicative of one or more biomarkers in the mixture.

Aspects of the present disclosure are directed to a method of providing point-of-care screening of a patient. In some embodiments, the method includes collecting a patient sample; mixing the patient sample with an electroactive solution including a concentration of metal ions to form a sample mixture; contacting an amount of the sample mixture with a pair of electrodes; performing an electrochemical analysis of the sample mixture via the pair of electrodes to produce an electrochemical data output; identifying a distinct electrochemical signature in the electrochemical data output indicative of one or more biomarkers in the sample mixture; diagnosing a patient with a condition based on the distinct electrochemical signature; and administering a treatment to the patient effective to treat the condition.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic representation of a system for point-of-care screening of a patient for a condition according to some embodiments of the present disclosure;

FIG. 2 is a schematic representation of a sample collector for collecting patient samples according to some embodiments of the present disclosure;

FIG. 3 is a chart of a method of providing point-of-care screening of a patient according to some embodiments of the present disclosure;

FIG. 4A is a graph portraying square wave voltammetry (SWV) analysis of a tuberculosis-positive patient sample using systems and methods according to embodiments of the present disclosure;

FIG. 4B is a graph portraying SWV analysis of a tuberculosis-negative patient sample using systems and methods according to embodiments of the present disclosure;

FIG. 4C is a graph portraying SWV analysis of a tuberculosis-negative patient sample using systems and methods according to embodiments of the present disclosure;

FIG. 5A is a graph showing electrochemical signatures identified for a solution including methyl nicotinate (MN) biomarker according to some embodiments of the present disclosure;

FIG. 5B is a graph showing electrochemical signatures identified for a solution including MN biomarker according to some embodiments of the present disclosure;

FIG. 6A is a graph portraying training data receiver operator characteristic (ROC) results for a machine learning algorithm according to some embodiments of the present disclosure;

FIG. 6B is a graph portraying testing data ROC results for a machine learning algorithm according to some embodiments of the present disclosure;

FIG. 6C is a graph portraying feature importance of the SWV analysis from FIG. 4A as defined by a machine learning algorithm according to some embodiments of the present disclosure;

FIG. 7 is a flowchart portraying systems and methods for screening patients for a condition according to some embodiments of the present disclosure.

### DETAILED DESCRIPTION

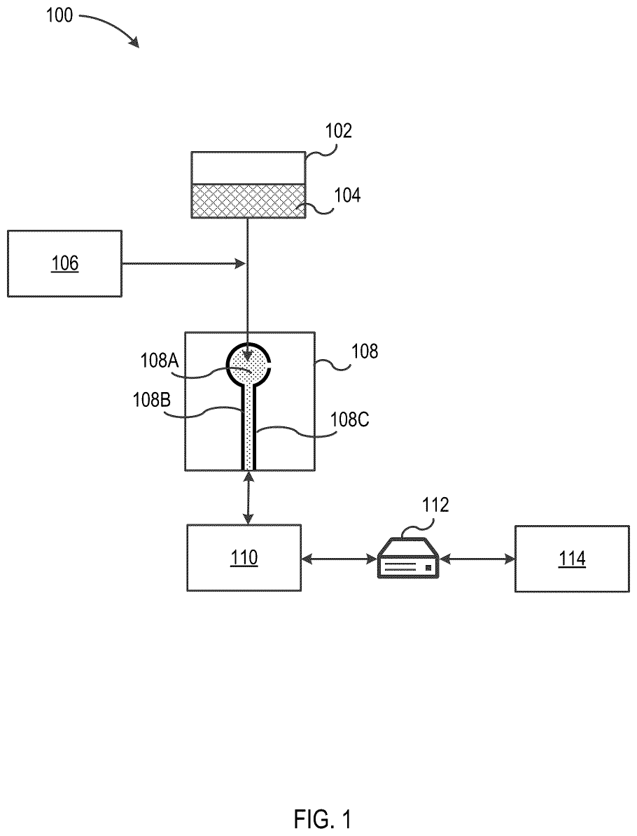

Referring now to FIG. 1, some embodiments of the present disclosure are directed to a system 100 for point-of-care screening of a patient for a condition. In some embodiments, system 100 is configured for electrochemically detecting one or more biomarkers, also referred to herein as volatile organic biomarkers (VOBs) or volatile organic compounds (VOCs).

In some embodiments, the one or more biomarkers are present in a patient sample, e.g., a sample obtained from the patient of interest, and indicative of a positive diagnosis of the patient for a disease/condition. In some embodiments, the patient sample includes one or more liquids, gases, or combinations thereof. In some embodiments, the patient sample includes exhaled gases from a human patient. In some embodiments, the patient sample includes breath, urine, saliva, sweat, odor, or combinations thereof, from the patient. In some embodiments, the biomarkers are for any desired condition. In some embodiments, the one or more biomarkers are infectious disease biomarkers, cancer biomarkers, non-communicable disease biomarkers, or combinations thereof. In some embodiments, the biomarkers are tuberculosis (TB) biomarkers, pneumonia biomarkers, malaria biomarkers, schistosomiasis biomarkers, colorectal cancer biomarkers, or combinations thereof.

In some embodiments, system 100 includes a solution reservoir 102 including a volume of an electroactive solution 104. In some embodiments, electroactive solution 104 is an aqueous solution. In some embodiments, electroactive solution 104 includes an organic solution. In some embodiments, electroactive solution 104 includes acetone, ethanol, I⁺, citric acid, acetic acid, or combinations thereof. In some embodiments, electroactive solution 104 includes one or more metal ions and/or metal-containing salts. In some embodiments, electroactive solution 104 includes acetone, ethanol, I⁺ (iodine), citric acid, acetic acid, metals ions, or combinations thereof. In some embodiments, electroactive solution 104 includes acetone, ethanol with I⁺, citric acid, and metals ions. In some embodiments, electroactive solution 104 includes acetone, ethanol with I⁺, acetic acid, and metals ions.

In some embodiments, the metal ions in electroactive solution 104 include ions of Li, Cu, Fe, Ni, Cr, Co, Ca, Mg, Zn, In, Al, or combinations thereof. In some embodiments, electroactive solution 104 includes Li¹⁺, Cu¹⁺, Fe²⁺, Ni²⁺, Cr²⁺, Co²⁺, Ca²⁺, Mg²⁺, Zn²⁺, Cu²⁺, In³⁺, Co³⁺, Fe³⁺, Al³⁺, Cr³⁺, or combinations thereof. In some embodiments, electroactive solution 104 includes lithium metal salts, copper metal salts, iron metal salts, nickel salts, chromium metal salts, cobalt metal salts, calcium metal salts, magnesium metal salts, zinc metal salts, indium metal salts, aluminum metal salts, or combinations thereof. In some embodiments, electroactive solution 104 includes lithium (I) metal salts, copper (I) metal salts, iron (II) metal salts, nickel (II) salts, chromium (II) metal salts, cobalt (II) metal salts, calcium (II) metal salts, magnesium (II) metal salts, zinc (II) metal salts, copper (II) metal salts, indium (III) metal salts, cobalt (III) metal salts, iron (III) metal salts, aluminum (III) metal salts, chromium (III) metal salts, or combinations thereof. In some embodiments, electroactive solution 104 includes copper (II) chloride.

Referring again to FIG. 1, system 100 includes a sample collector 106. Sample collector 106 is configured to collect a patient sample directly, e.g., from a human patient. In some embodiments, sample collector 106 is configured to transport the patient sample to electroactive solution 104. In some embodiments, sample collector 106 is configured to collect one or more liquids, gases, or combinations thereof from a patient. In some embodiments, sample collector 106 is configured to collect breath, urine, saliva, sweat, odor, or combinations thereof, from a patient. In some embodiments, sample collector 106 collects patient sample 106 passively, i.e., the patient sample is provided by the patient to and accumulates in and/or on the sample collector over a period of time. In some embodiments, sample collector 106 includes a mask, an absorbent substrate, a conduit, or combinations thereof.

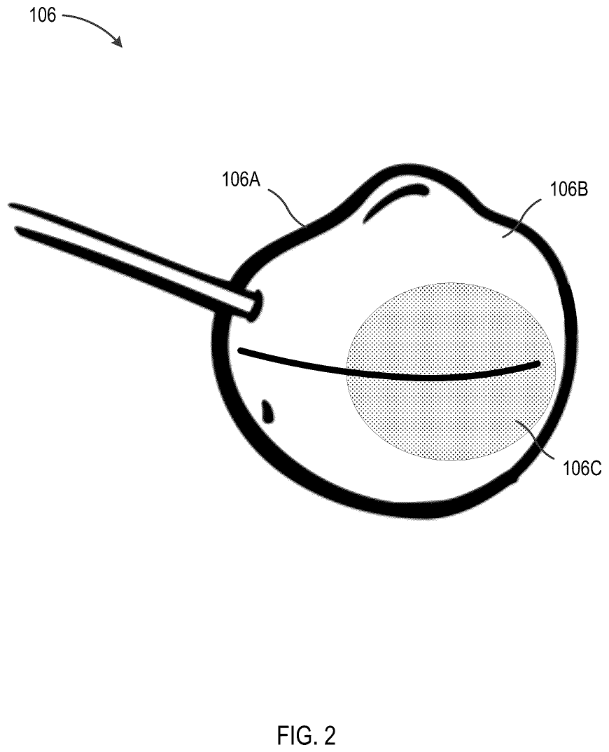

Referring now to FIG. 2, in some embodiments, sample collector 106 includes a mask 106A. In some embodiments, mask 106A is configured to be worn over a patient's nose, mouth, or combinations thereof. In some embodiments, mask 106A is a standard mask. In some embodiments, mask 106A is composed of one or more polymeric materials. In some embodiments, mark 106A includes a substrate 106B positioned to collect patient sample from the patient's nose, mouth, or combinations thereof. In some embodiments, substrate 106B is a portion of mask 106A itself. In some embodiments, at least a portion of substrate 106B is composed of a different material than the remainder of mask 106A. In some embodiments, substrate 106B includes an absorbent material. In some embodiments, at least a portion of substrate 106B is reversibly attached to mask 106A.

In some embodiments, mask 106A is configured to direct patient sample collected directly from the patient to substrate 106B. In some embodiments, mask 106A operates as a passive biomarker/VOC absorbent medium. In these embodiments, patient sample accumulates on substrate 106B, e.g., directly on mark 106A itself, on a dedicated absorptive portion thereof, etc., over a period of time while worn by the user. In some embodiments, the period of time can be between about 20 minutes and about 60 minutes. In some embodiments, accumulated patient sample is recovered from substrate 106B. In some embodiments, the area of mask 106A/substrate 106B on which the patient sample accumulated is removed, e.g., by cutting a defined region from mask 106A itself, removing an absorbent substrate 106B from the mask, etc. The removed area, e.g., 106C, which contains accumulated patient sample, can then be contacted directly with electroactive solution, or the accumulated patient sample on the removed area can be recovered and separately contacted with electroactive solution, as will be discussed in greater detail below.

Referring again to FIG. 1, system 100 includes electrodes 108. In some embodiments, electrodes 108 are screen-printed. In some embodiments, electrodes 108 include a pair of electrodes, e.g., a working electrode 108A and a counter electrode 108B. In some embodiments, working electrode 108A and/or counter electrode 108B are composed of any suitable material for use in liquid-phase electrochemical analysis of patient sample, e.g., carbon. In some embodiments, electrodes 108 also include a reference electrode 108C. In some embodiments, electrodes 108 are configured to be contacted by a sample mixture including an amount of the patient sample and electroactive solution 104. In some embodiments, electrodes 108 are in fluid communication with solution reservoir 102. In some embodiments, electrodes 108 are maintained in contact with electroactive solution 104. In these embodiments, a patient sample, e.g., accumulated on removed area 106C, can be immersed in electroactive solution 104, enabling desorption of patient sample and captured VOCs into the liquid phase, forming a sample mixture in electrical contact with electrodes 108. Liquid-phase detection, rather than gas-phase detection, is preferable for semi-volatile compounds such as many TB biomarkers. Without wishing to be bound by theory, this approach can offer more consistent results due to the presence of a counter electrode, which reduces the impact of production variability from the working electrode.

In some embodiments, system 100 includes a potentiostat 110 in electrical contact with electrodes 108. In some embodiments, system 100 is operated with a commercially available potentiostat (PalmSens) that may be controlled, e.g., by a smartphone via Bluetooth for automated analysis and read-out. In some embodiments, system 100 is configured to perform electrochemical analysis on the sample mixture via electrodes 108. In some embodiments, system 100 is configured to perform electrochemical analysis on electroactive solution after a patient sample has been mixed therein. In some embodiments, the electrochemical analysis produces an electrochemical data output corresponding to the analysis being performed. In some embodiments, the electrochemical analysis includes amperometry, potentiometry, conductometry, square-wave voltammetry (SWV), or combinations thereof.

In some embodiments, the electrochemical data output includes an electrochemical signature indicative of the composition of the medium being analyzed, e.g., an electroactive solution “blank,” the sample mixture, etc. The systems and methods according to embodiments of the present disclosure can detect trace amounts of analytes, such as VOBs, while minimizing background interference, making it a promising diagnostic approach, e.g., for TB diagnosis. SWV, in particular, is an electroanalytical technique that applies a series of square wave potentials to an electrode, resulting in improved sensitivity and selectivity. In some embodiments, biomarker signal, e.g., electrochemical signatures, is amplified to enhance the sensitivity of system 100.

Distinct electrochemical signatures are indicative of one or more biomarkers in the sample mixture. Furthermore, the presence of the biomarkers in the patient sample, confirmed by the distinct electrochemical signatures thereof, can be used to diagnose the patient with a particular condition associated with those biomarkers. By way of example, a patient with TB gives off VOCs methyl nicotinate (MN) and methyl p-anisate (MPA) that are a direct metabolic product of the mycobacterium. The patient's breath can be collected via a condensate collector, e.g., the mask discussed above. The condensate can then be mixed with an electroactive solution with a concentration of metal ions or metal-containing salts, e.g., cobalt or copper (II), e.g., by excising removed area 106C and immersing it in the solution. At least a drop of the solution can then be placed on screen-printed electrodes for electrochemical analysis using SWV. The VOCs undergo redox reactions, generating electrochemical signatures that serve as “fingerprints” for disease-specific biomarkers. Thus, the resulting SWV curves yield a distinct electrochemical signature indicative of the presence of the MN and MPA, and enable a confident diagnosis of TB in the patient.

In some embodiments, the one or more biomarkers includes MN; MPA; 2-methyl butane; acrolein; furan; tetrahydro; heptane; ethylbenzene; carane; tetradecane; tetradecanal; octane; hexanal, 2,4-dimethylheptane; ethylcyclohexane; 2,4-dimethylhept-1-ene; 4-hydroxy-4-methylpentan-2-one; ethylbenzene, m-xylene; p-xylene; o-xylene; propylcyclohexane; 1-ethyl-3-methylbenzene; benzaldehyde; 1,2,4-trimethylbenzene; decane; octanal; s(−)-limonene; 2-ethylhexan-1-ol; nonanal; dodecane; phenylacetylglutamine (PAG); hippurate; 1,1-(1-butenylidene)-bis-benzene; 1,3-dimethylbenzene; 1,4-dimethylbenzene; 2-amino-5-isopropyl-8-methyl-1-azulenecarbonitrile; 1-iodononane; [(1,1-dimethylethyl)thio]acetic acid; 4-(4-propylcyclohexyl)-40-cyano[1,10-bibiphenyl]-4-yl ester benzoic acid; decanal; 4-methyl-2-pentanone; 2-pentanone; 4-methyloctane; 4-methylundecane; 2-methylpentane; 3-methylpentane; methylcyclopentane; cyclohexane; methylcyclohexane; trimethyldecane-1,2-pentadiene; cyclohexanone; 2,2-dimethyldecane; 4-ethyl-1-octyn-3-ol; trans-2-dodecen-1-ol; cyclooctylmethanol; ethylaniline; heptanal; 2-methyl-3-phenyl-2-propenal; p-cymene; 1,2-dihydro-1,1,6-trimethyl-naphthalene; 1,4,5-trimethylnaphthalene; anisole; 1-octanol; 4-methylphenol; c-terpinene; bomylene; dimethyl disulfide; 2-methoxythiophene; 2,7-dimethylquinoline; acetaldehyde; acetone; 4-heptanone; allylisothiocyanate; methoxy-phenyl-oxime; hexen-1-ol, or combinations thereof.

Still referring to FIG. 1, in some embodiments, system 100 includes a computer readable storage device 112 having stored thereon instructions 114. In some embodiments, when executed by one or more processors, instructions 114 train a machine learning algorithm. In some embodiments, the machine learning algorithm is trained on an electrochemical training data set to identify an electrochemical signature indicative of a particular biomarker. In some embodiments, the electrochemical training data set includes a plurality of electrochemical data outputs from condition-positive patient samples and electrochemical data outputs from condition-negative patient samples. In some embodiments, the electrochemical training data set includes data obtained from a process including amperometry, potentiometry, conductometry, SWV, or combinations thereof.

In exemplary embodiments, the machine learning algorithm of system 100 includes the “XGBoost” model, modified with Optuna and with overtraining avoided. XGBoost can be used and is designed to handle imbalanced data sets. XGBoost can construct a sequence of decision trees, with each successive tree built to correct errors found in the previous tree, enabling the model to learn from inaccurate predictions. The objective function of the XGBoost model can be set to minimize the difference between predicted and actual values through gradient descent.

XGBoost is a gradient-boosting model which, due to its adaptive complexity, can obfuscate features impactful on its predictions. XGBoost can also be prone to overfitting. Overtraining occurs when the model matches artifacts in the training data too closely, leading to a lack of generalizability when applied to new data. Embodiments of the present disclosure include hyperparameter tuning and cross-validation to mitigate this risk. To prevent overfitting, a penalty term can be assigned to the loss function, discouraging the model from fitting too closely to the training data. Tree pruning can also be employed, where the model splits up to the maximum depth specified and then prunes the tree backward, removing any splits beyond which no positive gain is achieved. In an exemplary embodiment, the Synthetic Minority Over-sampling Technique (SMOTE) application in Python was utilized to reduce the risk of overtraining. Balancing data with SMOTE to improve prediction accuracy across various algorithms has been demonstrated for the prediction of heart disease. Recursive feature elimination (RFE) and SMOTE has been applied to an imbalanced dataset to predict spinal disease, which showed that a SMOTE-RFE-XGBoost model could produce the best classification scores of the models tested.

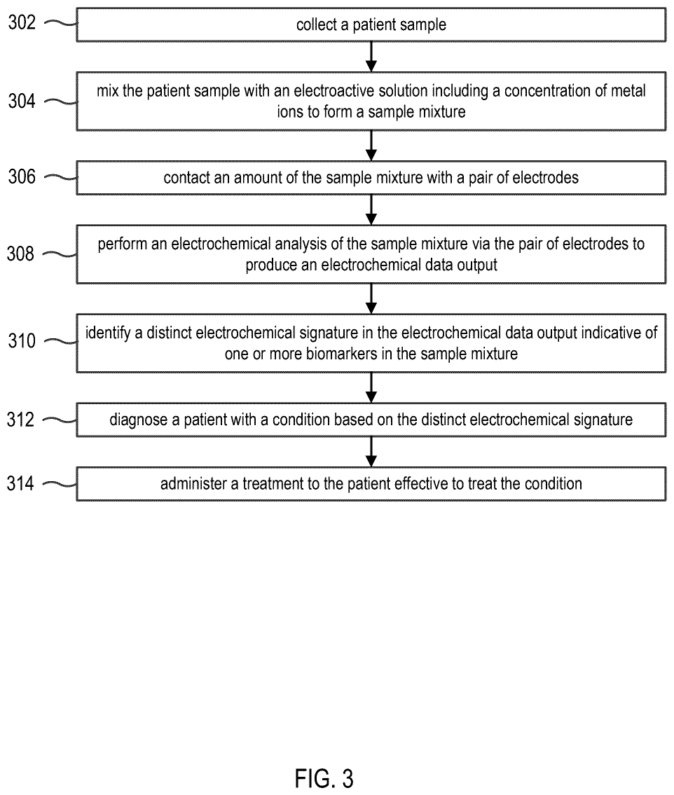

Referring now to FIG. 3, some embodiments of the present disclosure are directed to a method 300 of providing point-of-care screening of a patient. At 302, a patient sample is collected. As discussed above, in some embodiments, the patient sample is collected 302 via a sample collector. In some embodiments, the sample collector includes a mask, an absorbent substrate, a conduit, or combinations thereof. In some embodiments, the patient sample includes breath, urine, saliva, sweat, odor, or combinations thereof. In some embodiments of method 300, mask material acts as a passive VOC absorbent medium, allowing continuous accumulation over a period of, e.g., 20 to 60 minutes, while worn by the user.

At 304, the patient sample is mixed with an electroactive solution including a concentration of metal ions to form a sample mixture. In some embodiments, mixing 304 the patient sample with an electroactive solution includes immersing a portion of the sample collector in the electroactive solution.

As discussed above, in some embodiments, the electroactive solution is an aqueous solution. In some embodiments, the electroactive solution includes an organic solution. In some embodiments, the electroactive solution includes acetone, ethanol, I⁺, citric acid, acetic acid, or combinations thereof. In some embodiments, the electroactive solution includes one or more metal ions and/or metal-containing salts. In some embodiments, the electroactive solution includes acetone, ethanol, I⁺ (iodine), citric acid, acetic acid, metals ions, or combinations thereof. In some embodiments, the electroactive solution includes acetone, ethanol with I⁺, citric acid, and metals ions. In some embodiments, the electroactive solution includes acetone, ethanol with I⁺, acetic acid, and metals ions. In some embodiments, the metal ions in the electroactive solution include ions of Li, Cu, Fe, Ni, Cr, Co, Ca, Mg, Zn, In, Al, or combinations thereof. In some embodiments, the electroactive solution includes Li¹⁺, Cu¹⁺, Fe²⁺, Ni²⁺, Cr²⁺, Co²⁺, Ca²⁺, Mg²⁺, Zn²⁺, Cu²⁺, In³⁺, Co³⁺, Fe³⁺, Al³⁺, Cr³⁺, or combinations thereof. In some embodiments, the electroactive solution includes lithium metal salts, copper metal salts, iron metal salts, nickel salts, chromium metal salts, cobalt metal salts, calcium metal salts, magnesium metal salts, zinc metal salts, indium metal salts, aluminum metal salts, or combinations thereof. In some embodiments, the electroactive solution includes lithium (I) metal salts, copper (I) metal salts, iron (II) metal salts, nickel (II) salts, chromium (II) metal salts, cobalt (II) metal salts, calcium (II) metal salts, magnesium (II) metal salts, zinc (II) metal salts, copper (II) metal salts, indium (III) metal salts, cobalt (III) metal salts, iron (III) metal salts, aluminum (III) metal salts, chromium (III) metal salts, or combinations thereof. In some embodiments, the electroactive solution includes copper (II) chloride.

Still referring to FIG. 3, at 306, an amount of the sample mixture is contacted with a pair of electrodes. In some embodiments, mixing 304 is first performed to form the sample mixture, and the sample mixture is then subsequently contacted 306. In some embodiments, the electroactive solution is first contacted with the electrodes, and the patient sample is then mixed 304 while the electroactive solution is in contact with the electrodes (resulting in contact 306). In some embodiments, the patient sample is first contacted with the electrodes, and the electroactive solution is then mixed 304 while the patient sample is in contact with the electrodes (again, resulting in contact 306).

At 308, an electrochemical analysis of the sample mixture is performed via the pair of electrodes to produce an electrochemical data output. In some embodiments, the electrochemical analysis includes amperometry, potentiometry, conductometry, SWV, or combinations thereof. At 310, a distinct electrochemical signature in the electrochemical data output is identified. In some embodiments, identifying 310 the distinct electrochemical signature in the electrochemical data output indicative of one or more biomarkers in the sample mixture includes training a machine learning algorithm on an electrochemical training data set to identify electrochemical signatures indicative of a particular biomarker. As discussed above, in some embodiments, the electrochemical training data set includes a plurality of electrochemical data outputs from condition-positive patient samples and electrochemical data outputs from condition-negative patient samples. In some embodiments, the electrochemical training data set includes data obtained from a process including amperometry, potentiometry, conductometry, SWV, or combinations thereof.

As discussed above, in some embodiments, the distinct electrochemical signature is indicative of one or more biomarkers in the sample mixture. In some embodiments, the one or more biomarkers includes MN; MPA; 2-methyl butane; acrolein; furan; tetrahydro; heptane; ethylbenzene; carane; tetradecane; tetradecanal; octane; hexanal; 2,4-dimethylheptane; ethylcyclohexane; 2,4-dimethylhept-1-ene; 4-hydroxy-4-methylpentan-2-one; ethylbenzene; m-xylene; p-xylene; o-xylene; propylcyclohexane; 1-ethyl-3-methylbenzene; benzaldehyde; 1,2,4-trimethylbenzene; decane; octanal; s(−)-limonene; 2-ethylhexan-1-ol; nonanal; dodecane; phenylacetylglutamine (PAG); hippurate; 1,1-(1-butenylidene)-bis-benzene; 1,3-dimethylbenzene; 1,4-dimethylbenzene; 2-amino-5-isopropyl-8-methyl-1-azulenecarbonitrile; 1-iodononane; [(1,1-dimethylethyl)thio]acetic acid; 4-(4-propylcyclohexyl)-40-cyano[1,10-bibiphenyl]-4-yl ester benzoic acid; decanal; 4-methyl-2-pentanone; 2-pentanone; 4-methyloctane; 4-methylundecane; 2-methylpentane; 3-methylpentane; methylcyclopentane; cyclohexane; methylcyclohexane; trimethyldecane-1,2-pentadiene; cyclohexanone; 2,2-dimethyldecane; 4-ethyl-1-octyn-3-ol; trans-2-dodecen-1-ol; cyclooctylmethanol; ethylaniline; heptanal; 2-methyl-3-phenyl-2-propenal; p-cymene; 1,2-dihydro-1, 1,6-trimethyl-naphthalene; 1,4,5-trimethylnaphthalene; anisole; 1-octanol; 4-methylphenol; c-terpinene; bomylene; dimethyl disulfide; 2-methoxythiophene; 2,7-dimethylquinoline; acetaldehyde; acetone; 4-heptanone; allylisothiocyanate; methoxy-phenyl-oxime; hexen-1-ol, or combinations thereof.

In some embodiments, at 312, a patient is diagnosed with a condition based on the distinct electrochemical signature. In some embodiments, the condition includes TB, pneumonia, malaria, schistosomiasis, colorectal cancers, or combinations thereof. In some embodiments, at 314, a treatment effective to treat that condition is administered to the patient.

In some embodiments, breath compounds are eliminated from the sample during and/or before electrochemical testing, at 308. In some embodiments, the breath compounds that are eliminated are those that can and/or will produce competing reactions in the electroactive solution. In some embodiments, the breath compounds are eliminated via any suitable means, e.g., via leveraging differing solubilities of the compounds. By modifying these processes, the electrochemical signal can detect lower concentrations, e.g., of TB biomarkers, and potentially improving diagnostic accuracy.

In an example of some embodiments of the present disclosure, aqueous solutions were prepared using deionized water (≥18MΩcm—1 type 1 ultrapure, PURELAB Classic). Base electroactive solutions included aqueous copper (II) chloride (Alfa Aesar, anhydrous, 98% min) as the source of active metal ions and sodium chloride (Fisher Chemical, ≥99.0%) as a supporting electrolyte with concentrations of 1 mM copper chloride and 100 mM sodium chloride.

57 patient breath samples, including 26% negative for TB and 74% confirmed TB were collected in 10-liter Tedlar® Breath Analysis Bags (SKC Inc., USA) at Makerere University in Uganda and pumped through the copper salt solution at a rate of approximately 1 L/min. This breath-solution transfer device was designed to eliminate sample contamination by pulling gas from the bag into the solution. This was accomplished by placing the pump at the end of the flow path after the breath sample had been pulled into the salt solution.

The salt solution with dissolved breath was applied to a screen-printed electrode surface with a volume of 150 μL. A SWV method was then run with the settings seen in Table 1 below. Liquid solutions were electrochemically analyzed using a hand-held potentiostat (PalmSens, EmStat) and screen-printed electrode with unmodified carbon working/counter electrodes and a silver reference electrode (DRP110, 4 mm diameter WE, Metrohm-DropSens).

TABLE 1
Settings for Square Wave Voltammetry Method

| Parameter | Value | |
|---|---|---|
| t equilibration | 5 | seconds |
| E begin | −0.5 | V |
| E end | 0.4 | V |
| E step | 0.005 | V |
| Amplitude | 0.025 | V |
| Frequency | 2 | Hz |

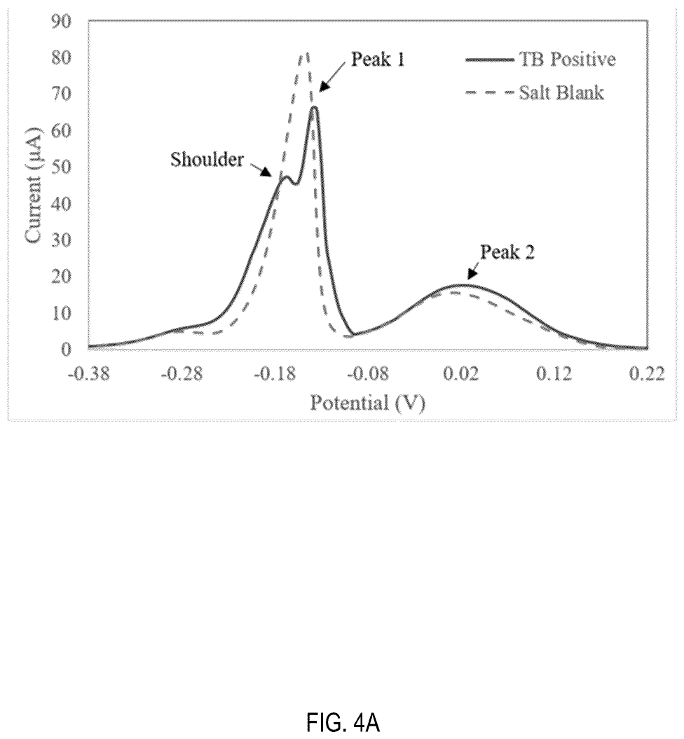

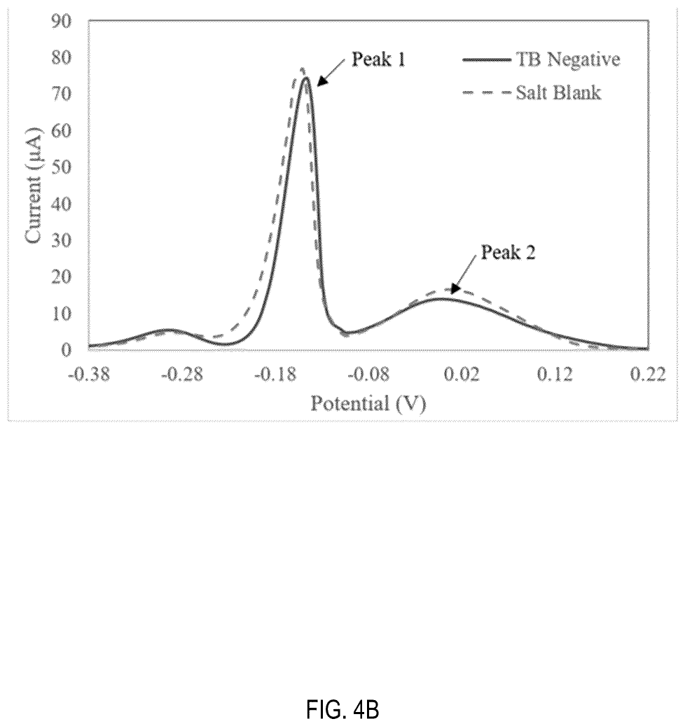

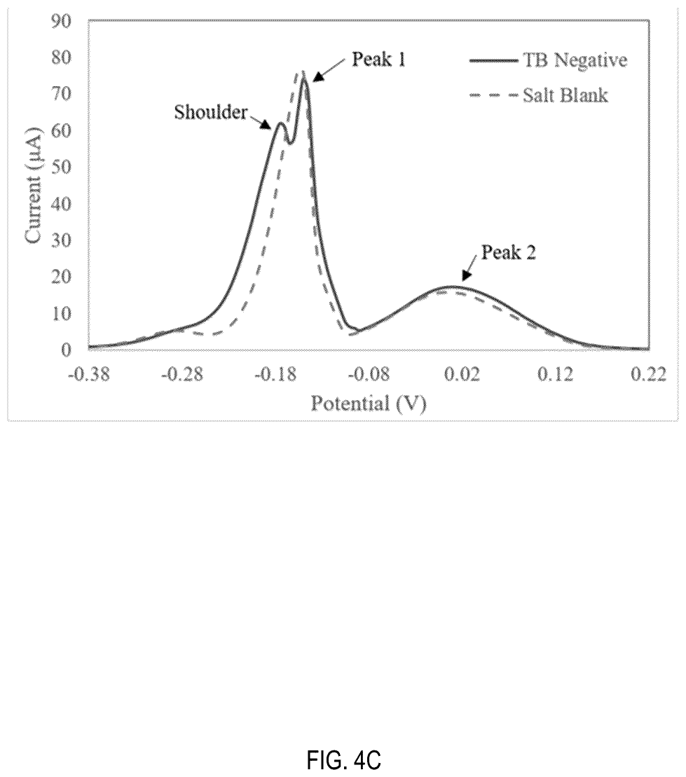

Referring now to FIGS. 4A-4C, SWV current values for a daily salt blank were obtained and compared to SWV results for the breath solution for each patient. The salt blank was used to establish a baseline current response for the Cu (II) salt solution to eliminate the effects of slight variation in salt concentration across the patient sampling period. Results for each patient sample relative to the blank solution were plotted and visually examined.

Three features were identified that correlate with TB status. These features represent specific oxidation/reduction reactions that occur for Cu under SWV. For example, peak 1 represents Cu²⁺↔Cu⁰ and peak 2 represents Cu⁺↔Cu⁰. Without wishing to be bound by theory, the current associated with these peaks reduces in relation to MN concentration.

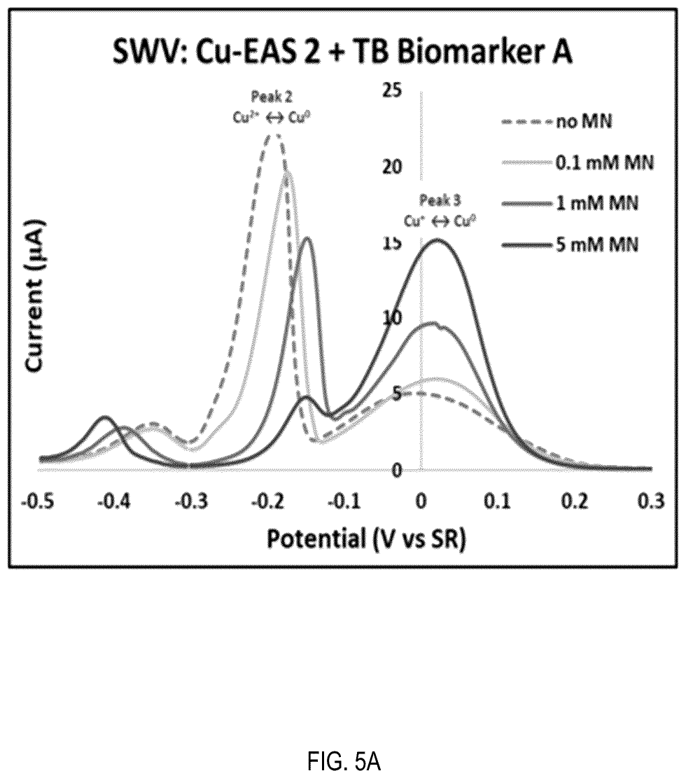

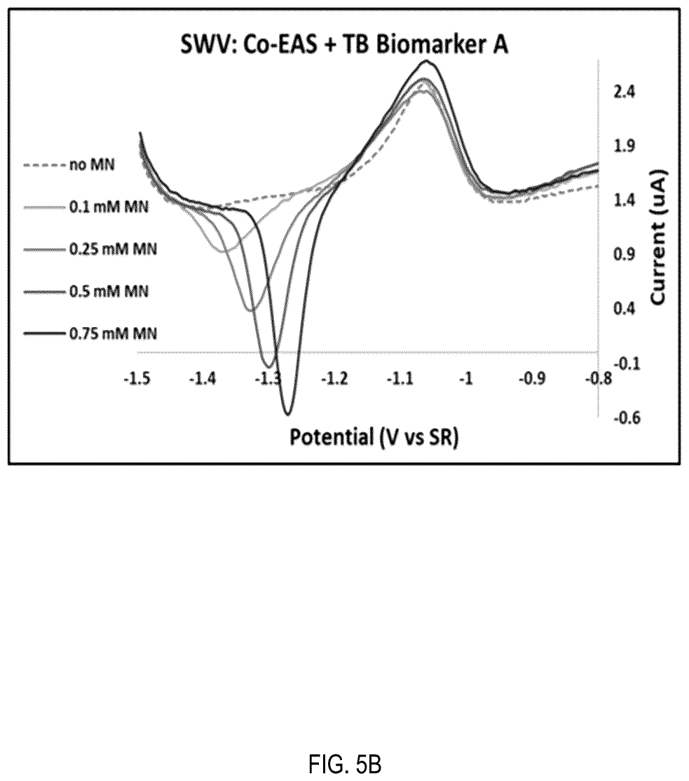

Referring now to FIG. 5A-5B, to further demonstrate the identification of electrochemical signatures for the detection of biomarkers, additional SWV processes were performed on electroactive solutions including varying concentrations of MN. In the copper salt solution (FIG. 5A), decreases in peak 2 and increases peak 3 were observed, indicating an interaction between different copper valent states and MN. For the cobalt salt (FIG. 5B), another peak shows up in the −1.35 to −1.2 V range as the concentration of MN increases. Combined these curves provide an electrochemical profile that can be used to detect MN in condensate.

Referring again to FIGS. 4A-4C, to further elucidate current behavior when breath samples contained MN, spiked mimics were created by taking 24 healthy breath samples and transferring them to the metal salt solution. After these known concentrations of MN were added to the liquid solution, a distinct “shoulder peak” at a low concentration of MN (approximately 0.1 mM) appeared similar to patients with confirmed TB (as seen in FIG. 4A). Additionally, a consistent reduction in current was seen in the presence of MN at peak 1 for both lab mimics and patient data. Leveraging the molecular structures of MN and MPA, the exemplary embodiment selectively detected these biomarkers, effectively reducing interference from other VOBs present in breath samples.

The current values normalized to peak maxima in the salt blank data for the “shoulder” feature and peaks 1 and 2 were then extracted. The distribution of Confirmed TB patients to TB negative was imbalanced (26% TB negative and 74% Confirmed TB). To address this issue, the SMOTE application in Python was used to balance the data set prior to machine learning analysis. With SMOTE, the sample comprised 32 negative and 31 positive samples for the training data and 10 negative and 11 positive in the testing data. This now balanced dataset was then used to train an XGBoost model.

XGBoost constructed a sequence of decision trees, with each successive tree built to correct errors found in the previous tree, enabling the model to learn from inaccurate predictions. The objective function of the XGBoost model was set to minimize the difference between predicted and actual values through gradient descent. To prevent overfitting, a penalty term was assigned to the loss function. Tree pruning was also employed.

The XGBoost model parameters were determined using Optuna, as seen in Table 2 below. These values were found by conducting 60 trials and minimizing the loss function for each. The performance of the trained XGBoost model was evaluated using several metrics. First, the model was revised for accuracy, and a receiver operating characteristic (ROC) curve was generated. This curve, which plots the True Positive Rate (sensitivity) against the False Positive Rate (1-specificity) at various threshold settings, allowed visualization of the tradeoff between sensitivity and specificity for different decision thresholds.

TABLE 2
Hyperparameters used for accuracy.

| Hyperparameter | Value |
|---|---|
| Max Depth | 5 |
| Gamma | 0.1228747 |
| Min Child Weight | 1 |
| Learning Rate | 0.117030 |
| Subsample | 1.0 |
| Colsample Bytree | 1.0 |
| Reg Alpha | 0.0197438 |
| Reg Lambda | 0.00017253 |
| Seed | 3 |

The efficacy of SWV was assessed using copper (II) metal salts for the detection of MN in the breath of the 57 adults in Uganda, which identified 26% patients negative for TB and 74% confirmed TB. The data was balanced using SMOTE split into a training set and a testing set, with 80% of the data used for training and 20% used for validation.

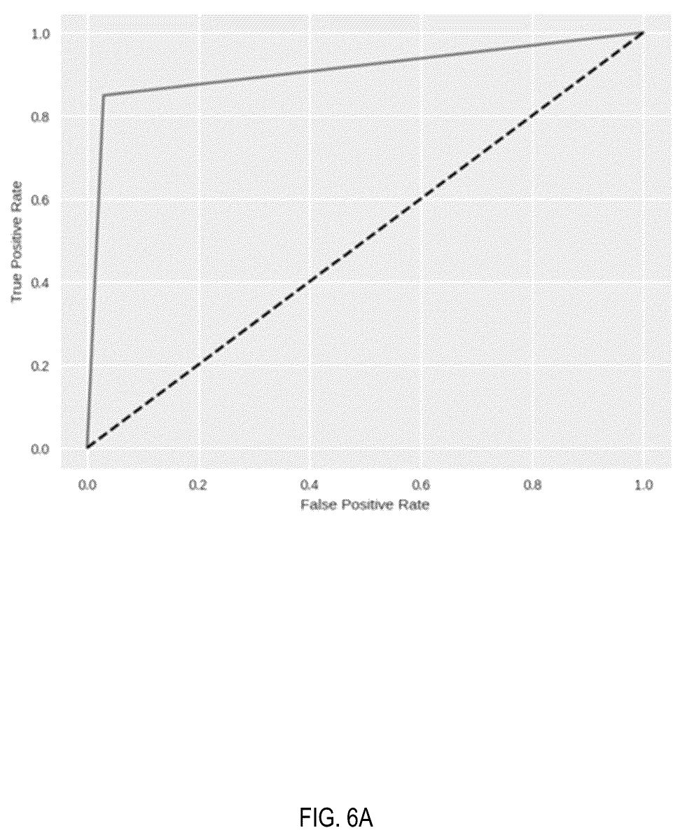

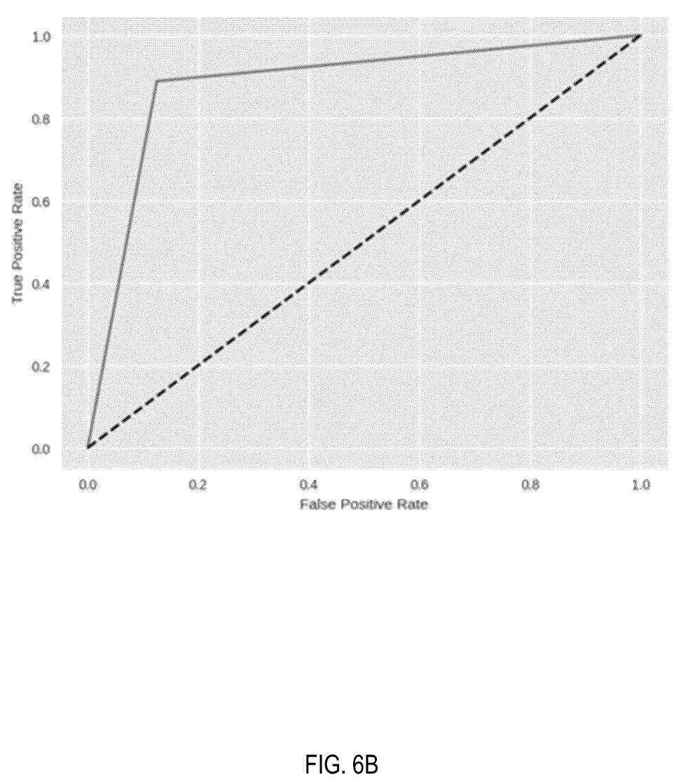

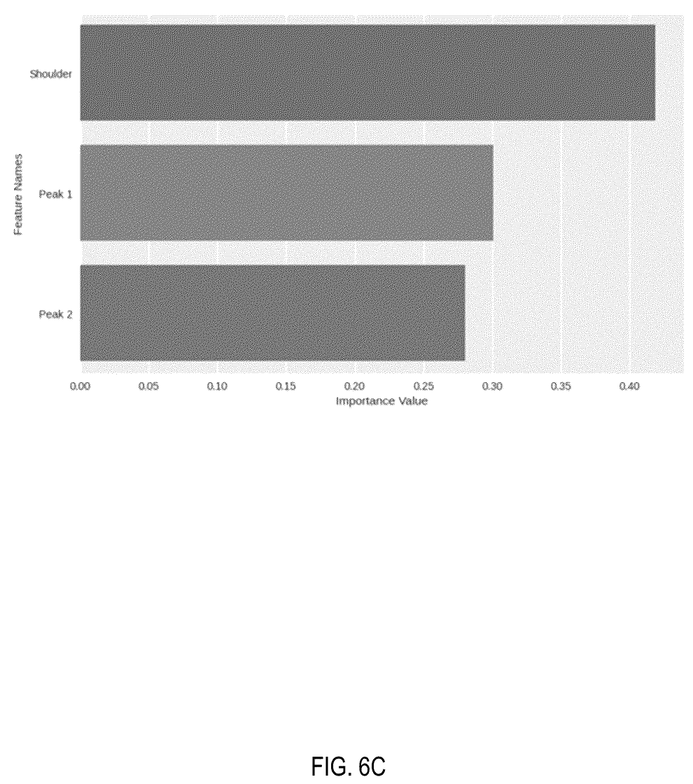

When correlated with TB diagnoses from sputum culture results, the sensor response was shown to have an accuracy of 0.88 and an area under the curve (AUC) value of 0.81 as shown in the ROC curve in FIG. 6A. The results for the training data are shown in FIG. 6B.

In addition to the AUC, sensitivity/selectivity was computed for both the training and test data, seen in Table 3. These values provide insight into how the algorithm is fitting the data, along with ensuring over or undertraining is minimized.

TABLE 3
Perfomance metrics for the exemplary model.

| Metric | Train | Test |
|---|---|---|
| Accuracy | 0.9104 | 0.8824 |
| Sensitivity | 0.8485 | 0.8888 |
| Selectivity | 0.9706 | 0.875 |
| AUC-ROC | 0.9684 | 0.8148 |

For the training data, the model achieved a sensitivity of 0.8571 and a selectivity of 0.9091. When applied to the testing data, the model yielded a sensitivity of 0.8888 and a selectivity of 0.875. These results suggest that this electrochemical technique has the potential to detect MN at the low concentration levels typically present in the breath of TB patients. Referring now to FIG. 6C, for the three features extracted for each patient, a “shoulder” feature appeared to have the greatest impact on status classification followed by peak 1.

Systems and methods of the present disclosure are advantageous to provide a rapid sensing platform for point-of-care detection of diseases such as TB, pneumonia, colorectal cancer, malaria, schistosomiasis, and others, as well as for detecting organic biomarkers in the field in a targeted way using low-cost tools.

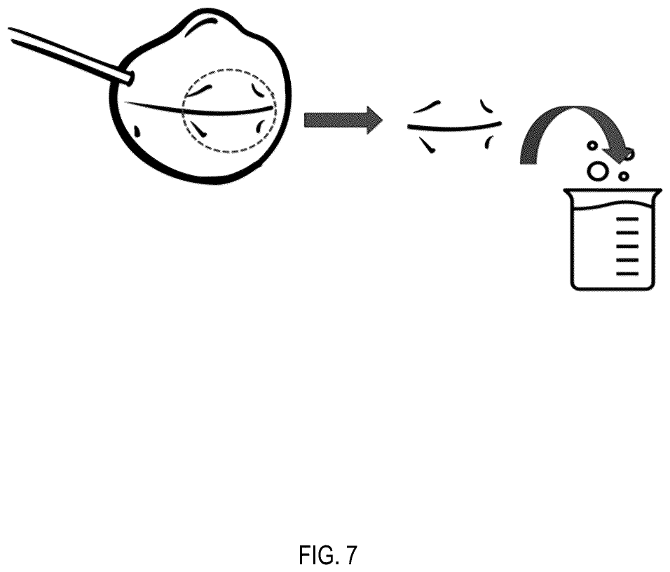

Referring specifically to FIG. 7, the systems and methods of the present disclosure provide a non-invasive diagnostic technique that can capture and analyze biomarkers, VOBs, VOCs, etc. from exhaled breath using a wearable mask-based sensor. The mask can be processed by cutting a defined region, for example about 2-inch in diameter, from the area corresponding to the mouth, ensuring advantageous concentration of exhaled biomarkers. This section can then be directly immersed in a clean determined electroactive solution, enabling the desorption of captured VOCs into liquid phase for electrochemical analysis. This technique eliminates the need for breath-bags sample collection procedure and simplifies the analysis process significantly.

Embodiments of the present disclosure were tested on a patient cohort comprised of 57 adults in Kampala, Uganda, of which 42 were confirmed TB-positive and 15 TB-negative. The example systems employed a copper (II) liquid metal salt solution with a SWV method tailored for MN detection using screen-printed electrodes. A machine learning analysis was performed using XGBoost, a type of gradient boosted Random Forest approach. Utilizing this approach, the sensor approached 89% sensitivity and 88% specificity with an accuracy score of 0.88. These results suggest the sensing methodology is effective in discriminating between TB-positive and TB-negative samples, providing a promising tool for non-invasive and rapid TB detection in clinical settings.

Given these results, the exemplary embodiments of the present disclosure show the applicability of this approach to advancements in TB disease diagnostics, with promising performance metrics that approach the WHO's target product profile for TB triage tests, providing early, rapid, and cost-effective diagnosis to millions who otherwise would not receive treatment. A traditional method of detection of these types of compounds is mass spectroscopy, which utilizes an expensive instrument. The systems and methods of the present disclosure do not require extensive training to use and are based on non-invasive sampling samples such as breath, urine, saliva, sweat, and odors.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A system for point-of-care screening of a patient for a condition, comprising:
a solution reservoir including a volume of an electroactive solution, the electroactive solution including a concentration of metal ions;
a sample collector configured to collect a patient sample directly from a human patient and transport the patient sample to the electroactive solution;
a pair of electrodes positioned to contact a sample mixture, the sample mixture including electroactive solution and patient sample; and
a potentiostat in electrical contact with the pair of electrodes,
wherein the system is configured to perform electrochemical analysis on the sample mixture via the pair of electrodes to produce an electrochemical data output including a distinct electrochemical signature indicative of one or more biomarkers in the sample mixture,
wherein the patient sample includes breath, urine, saliva, sweat, odor, or combinations thereof,
wherein the electrochemical analysis includes amperometry, potentiometry, conductometry, square-wave voltammetry (SWV), or combinations thereof, and
wherein the one or more biomarkers are infectious disease biomarkers, cancer biomarkers, non-communicable disease biomarkers, or combinations thereof.

2. The system according to claim 1, wherein the metal ions include Li, Cu, Fe, Ni, Cr, Co, Ca, Mg, Zn, In, Al, or combinations thereof.

3. The system according to claim 2, wherein the electroactive solution includes dissolved lithium (I) metal salts, copper (I) metal salts, iron (II) metal salts, nickel (II) salts, chromium (II) metal salts, cobalt (II) metal salts, calcium (II) metal salts, magnesium (II) metal salts, zinc (II) metal salts, copper (II) metal salts, indium (III) metal salts, cobalt (III) metal salts, iron (III) metal salts, aluminum (III) metal salts, chromium (III) metal salts, or combinations thereof.

4. The system according to claim 3, wherein the metal ions are provided by copper (II) chloride.

5. The system according to claim 1, wherein the electroactive solution further comprises acetone, ethanol, I⁺, citric acid, acetic acid, or combinations thereof.

6. The system according to claim 1, wherein the sample collector includes a mask, an absorbent substrate, a conduit, or combinations thereof.

7. The system according to claim 6, wherein the sample collector includes a mask with an absorbent substrate reversibly attached thereto, wherein the mask is configured to direct patient sample collected directly from the patient to the absorbent substrate.

8. The system according to claim 1, wherein the one or more biomarkers includes methyl-nicotinate (MN); methyl p-anisate (MPA); 2-methyl butane; acrolein; furan; tetrahydro; heptane; ethylbenzene; carane; tetradecane; tetradecanal; octane; hexanal; 2,4-dimethylheptane; ethylcyclohexane; 2,4-dimethylhept-1-ene; 4-hydroxy-4-methylpentan-2-one; ethylbenzene; m-xylene; p-xylene; o-xylene; propylcyclohexane; 1-ethyl-3-methylbenzene; benzaldehyde; 1,2,4-trimethylbenzene; decane; octanal; s(−)-limonene; 2-ethylhexan-1-ol; nonanal; dodecane; phenylacetylglutamine (PAG); hippurate; 1,1-(1-butenylidene)-bis-benzene; 1,3-dimethylbenzene; 1,4-dimethylbenzene; 2-amino-5-isopropyl-8-methyl-1-azulenecarbonitrile; 1-iodononane; [(1, 1-dimethylethyl)thio]acetic acid; 4-(4-propylcyclohexyl)-40-cyano[1,10-bibiphenyl]-4-yl ester benzoic acid; decanal; 4-methyl-2-pentanone; 2-pentanone; 4-methyloctane; 4-methylundecane; 2-methylpentane; 3-methylpentane; methylcyclopentane; cyclohexane; methylcyclohexane; trimethyldecane-1,2-pentadiene; cyclohexanone; 2,2-dimethyldecane; 4-ethyl-1-octyn-3-ol; trans-2-dodecen-1-ol; cyclooctylmethanol; ethylaniline; heptanal; 2-methyl-3-phenyl-2-propenal; p-cymene; 1,2-dihydro-1,1,6-trimethyl-naphthalene; 1,4,5-trimethylnaphthalene; anisole; 1-octanol; 4-methylphenol; c-terpinene; bomylene; dimethyl disulfide; 2-methoxythiophene; 2,7-dimethylquinoline; acetaldehyde; acetone; 4-heptanone; allylisothiocyanate; methoxy-phenyl-oxime; hexen-1-ol, or combinations thereof.

9. The system according to claim 1, further comprising a computer readable storage device having stored thereon instructions that when executed by one or more processors result in the following operation:
training a machine learning algorithm on an electrochemical training data set to identify an electrochemical signature indicative of a particular biomarker, the electrochemical training data including a plurality of electrochemical data outputs from condition-positive patient samples and electrochemical data outputs from condition-negative patient samples,
wherein the electrochemical training data set includes data obtained from a process including amperometry, potentiometry, conductometry, square-wave voltammetry (SWV), or combinations thereof.

10. A method of providing point-of-care screening of a patient, comprising:
collecting a patient sample;
mixing the patient sample with an electroactive solution including a concentration of metal ions to form a sample mixture;
contacting an amount of the sample mixture with a pair of electrodes;
performing an electrochemical analysis of the sample mixture via the pair of electrodes to produce an electrochemical data output;
identifying a distinct electrochemical signature in the electrochemical data output indicative of one or more biomarkers in the sample mixture;
diagnosing a patient with a condition based on the distinct electrochemical signature; and
administering a treatment to the patient effective to treat the condition,
wherein the patient sample includes breath, urine, saliva, sweat, odor, or combinations thereof,
wherein the electrochemical analysis includes amperometry, potentiometry, conductometry, square-wave voltammetry (SWV), or combinations thereof, and
wherein the condition includes tuberculosis (TB), pneumonia, malaria, schistosomiasis, colorectal cancers, or combinations thereof.

11. The method according to claim 10, wherein collecting the patient sample includes collecting the patient sample on a sample collector, wherein the sample collector includes a mask, an absorbent substrate, a conduit, or combinations thereof.

12. The method according to claim 11, wherein mixing the patient sample with an electroactive solution includes immersing a portion of the sample collector in the electroactive solution.

13. The method according to claim 10, wherein identifying the distinct electrochemical signature in the electrochemical data output indicative of one or more biomarkers in the sample mixture includes:
training a machine learning algorithm on an electrochemical training data set to identify electrochemical signatures indicative of a particular biomarker, the electrochemical training data including a plurality of electrochemical data outputs from condition-positive patient samples and electrochemical data outputs from condition-negative patient samples,
wherein the electrochemical training data set includes data obtained from a process including amperometry, potentiometry, conductometry, square-wave voltammetry (SWV), or combinations thereof.

14. The method according to claim 10, wherein the metal ions include Li, Cu, Fe, Ni, Cr, Co, Ca, Mg, Zn, In, Al, or combinations thereof.

15. The method according to claim 14, wherein the electroactive solution includes dissolved lithium (I) metal salts, copper (I) metal salts, iron (II) metal salts, nickel (II) salts, chromium (II) metal salts, cobalt (II) metal salts, calcium (II) metal salts, magnesium (II) metal salts, zinc (II) metal salts, copper (II) metal salts, indium (III) metal salts, cobalt (III) metal salts, iron (III) metal salts, aluminum (III) metal salts, chromium (III) metal salts, or combinations thereof.

16. The method according to claim 15, wherein the electroactive solution includes copper (II) chloride.

17. The method according to claim 10, wherein the electroactive solution further comprises acetone, ethanol, I⁺, citric acid, acetic acid, or combinations thereof.

18. The method according to claim 10, wherein the one or more biomarkers includes methyl-nicotinate (MN); methyl p-anisate (MPA); 2-methyl butane; acrolein; furan; tetrahydro; heptane; ethylbenzene; carane; tetradecane; tetradecanal; octane; hexanal; 2,4-dimethylheptane; ethylcyclohexane; 2,4-dimethylhept-1-ene; 4-hydroxy-4-methylpentan-2-one; ethylbenzene; m-xylene; p-xylene; o-xylene; propylcyclohexane; 1-ethyl-3-methylbenzene; benzaldehyde; 1,2,4-trimethylbenzene; decane; octanal; s(−)-limonene; 2-ethylhexan-1-ol; nonanal; dodecane; phenylacetylglutamine (PAG); hippurate; 1,1-(1-butenylidene)-bis-benzene; 1,3-dimethylbenzene; 1,4-dimethylbenzene; 2-amino-5-isopropyl-8-methyl-1-azulenecarbonitrile; 1-iodononane; [(1,1-dimethylethyl)thio]acetic acid; 4-(4-propylcyclohexyl)-40-cyano[1,10-bibiphenyl]-4-yl ester benzoic acid; decanal; 4-methyl-2-pentanone; 2-pentanone; 4-methyloctane; 4-methylundecane; 2-methylpentane; 3-methylpentane; methylcyclopentane; cyclohexane; methylcyclohexane; trimethyldecane-1,2-pentadiene; cyclohexanone; 2,2-dimethyldecane; 4-ethyl-1-octyn-3-ol; trans-2-dodecen-1-ol; cyclooctylmethanol; ethylaniline; heptanal; 2-methyl-3-phenyl-2-propenal; p-cymene; 1,2-dihydro-1,1,6-trimethyl-naphthalene; 1,4,5-trimethylnaphthalene; anisole; 1-octanol; 4-methylphenol; c-terpinene; bomylene; dimethyl disulfide; 2-methoxythiophene; 2,7-dimethylquinoline; acetaldehyde; acetone; 4-heptanone; allylisothiocyanate; methoxy-phenyl-oxime; hexen-1-ol, or combinations thereof.

19. A device for point-of-care screening of a patient, comprising:
a solution reservoir including a volume of an electroactive solution;
a pair of electrodes in contact with the electroactive solution;
a sample collector configured to collect a patient sample directly from a human patient and transport the patient sample to the electroactive solution, the sample collector including a mask with an absorbent substrate reversibly attached thereto, wherein the mask is configured to direct patient sample collected directly from the patient to the absorbent substrate; and
a potentiostat in electrical contact with the pair of electrodes,
wherein the device is configured to perform electrochemical analysis on the electroactive solution after the patient sample has been mixed therein via the pair of electrodes to produce an electrochemical data output including a distinct electrochemical signature indicative of one or more biomarkers in the mixture,
wherein the electroactive solution includes:
a dissolved concentration of lithium (I) metal salts, copper (I) metal salts, iron (II) metal salts, nickel (II) salts, chromium (II) metal salts, cobalt (II) metal salts, calcium (II) metal salts, magnesium (II) metal salts, zinc (II) metal salts, copper (II) metal salts, indium (III) metal salts, cobalt (III) metal salts, iron (III) metal salts, aluminum (III) metal salts, chromium (III) metal salts, or combinations thereof,
wherein the patient sample includes breath, urine, saliva, sweat, odor, or combinations thereof,
wherein the electrochemical analysis includes amperometry, potentiometry, conductometry, square-wave voltammetry (SWV), or combinations thereof, and
wherein the one or more biomarkers are tuberculosis (TB) biomarkers, pneumonia biomarkers, malaria biomarkers, schistosomiasis biomarkers, colorectal cancer biomarkers, or combinations thereof.

20. The device according to claim 19, wherein the one or more biomarkers includes methyl-nicotinate (MN); methyl p-anisate (MPA); 2-methyl butane; acrolein; furan; tetrahydro; heptane; ethylbenzene; carane; tetradecane; tetradecanal; octane; hexanal; 2,4-dimethylheptane; ethylcyclohexane; 2,4-dimethylhept-1-ene; 4-hydroxy-4-methylpentan-2-one; ethylbenzene; m-xylene; p-xylene; o-xylene; propylcyclohexane; 1-ethyl-3-methylbenzene; benzaldehyde; 1,2,4-trimethylbenzene; decane; octanal; s(−)-limonene; 2-ethylhexan-1-ol; nonanal; dodecane; phenylacetylglutamine (PAG); hippurate; 1,1-(1-butenylidene)-bis-benzene; 1,3-dimethylbenzene; 1,4-dimethylbenzene; 2-amino-5-isopropyl-8-methyl-1-azulenecarbonitrile; 1-iodononane; [(1, 1-dimethylethyl)thio]acetic acid; 4-(4-propylcyclohexyl)-40-cyano[1,10-bibiphenyl]-4-yl ester benzoic acid; decanal; 4-methyl-2-pentanone; 2-pentanone; 4-methyloctane; 4-methylundecane; 2-methylpentane; 3-methylpentane; methylcyclopentane; cyclohexane; methylcyclohexane; trimethyldecane-1,2-pentadiene; cyclohexanone; 2,2-dimethyldecane; 4-ethyl-1-octyn-3-ol; trans-2-dodecen-1-ol; cyclooctylmethanol; ethylaniline; heptanal; 2-methyl-3-phenyl-2-propenal; p-cymene; 1,2-dihydro-1,1,6-trimethyl-naphthalene; 1,4,5-trimethylnaphthalene; anisole; 1-octanol; 4-methylphenol; c-terpinene; bomylene; dimethyl disulfide; 2-methoxythiophene; 2,7-dimethylquinoline; acetaldehyde; acetone; 4-heptanone; allylisothiocyanate; methoxy-phenyl-oxime; hexen-1-ol, or combinations thereof.
